(19) United States
(12) Patent Application Publication
(10) Pub. No.: US 2004/0248932 A1
(43) Pub. Date: Dec. 9, 2004

(54) Use of aryl- and heteroaryl-substituted tetrahydroisoquinolines in the treatment of chronic and neuropathic pain, migraine headaches, and urge, stress and mixed urinary incontinence

(76) Inventors: Donald E. Frail, Wildwood, MO (US); Stephen P. Arneric, Milan, MI (US); Donn Gregory Wishka, Mystic, CT (US); Erik Ho Fong Wong, Portage, MI (US); James P. Beck, Zionsville, IN (US)

Correspondence Address: WARNER-LAMBERT COMPANY, 2800 PLYMOUTH RD, ANN ARBOR, MI, 48105, US

(21) Appl. No.: 10/724,856

(22) Filed: Dec. 1, 2003

Related U.S. Application Data
(60) Provisional application No. 60/430,285, filed on Dec. 2, 2002.

(51) Int. Cl. A61K 31/47

(52) U.S. Cl. 514/310

(57) ABSTRACT

Provided herein are methods of using compounds of formula (1) that are aryl- and heteroaryl-substituted tetrahydroisoquinolines, for the treatment of chronic and neuropathic pain, the treatment and prevention of migraine headache, and the treatment of stress, urge and mixed urinary incontinence.

### CROSS REFERENCE

This application claims the benefit of the following provisional application: U.S. Ser. No. 60/430,285 filed Dec. 2, 2002 under 35 USC 119(e)(i), which is incorporated herein by reference in its entirety

### FIELD OF THE INVENTION

The present invention relates to methods for the treatment of various disorders. In particular, the present invention relates to such methods wherein the compounds are novel 4-phenyl substituted tetrahydroisoquinolines derivatives.

### SUMMARY OF THE INVENTION

This invention is directed to the use of a compound of formula (1):

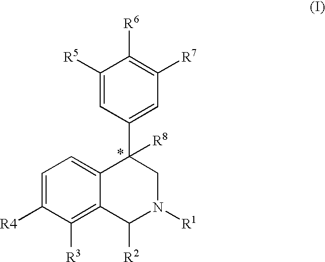

wherein:

the carbon atom designated * is in the R or S configuration;

R¹ is C₁-C₆ alkyl, C₂-C₆ alkenyl, C₂-C₆ alkynyl, C₃-C₆ cycloalkyl or C₄-C₇ cycloalkylalkyl, each of which is optionally substituted with from 1 to 3 substituents independently selected at each occurrence thereof from C₁-C₃ alkyl, halogen, Ar, —CN, —OR⁹ and —NR⁹R¹⁰;

R² is H, C₁-C₆ alkyl, C₂-C₆ alkenyl, C₂-C₆ alkynyl, C₃-C₆ cycloalkyl, C₄-C₇ cycloalkylalkyl or C₁-C₆ haloalkyl;

R³ is H, halogen, —OR¹¹, —S(O)ₙR¹², —CN, —C(O)R², —C(O)NR¹¹R¹², C₁-C₆ alkyl, C₂-C₆ alkenyl, C₂-C₆ alkynyl, C₃-C₆ cycloalkyl or C₄-C₇ cycloalkylalkyl and wherein each of C₁-C₆ alkyl, C₂-C₆ alkenyl, C₂-C₆ alkynyl, C₃-C₆ cycloalkyl and C₄-C₇ is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰;

R⁴ is aryl selected from phenyl, naphthyl and indenyl, or heteroaryl selected from pyridyl, pyrimidinyl, triazinyl, triazolyl, furanyl, pyranyl, indazolyl, benzimidazolyl, quinolinyl, quinazolinyl, isoquinolinyl, thienyl, imidazolyl, thiazolyl, benzthiazolyl, purinyl, isothiazolyl, indolyl, pyrrolyl, oxazolyl, benzofuranyl, benzothienyl, benzthiazolyl, isoxazolyl, pyrazolyl, oxadiazolyl and thiadiazolyl, wherein the aryl or heteroaryl group is optionally substituted with from 1 to 4 R¹⁴ substituents;

R⁵ and R⁶ and R⁷ are each independently H or are selected from halogen, —OR¹¹, —NR¹¹R¹², —NR¹¹C(O)R¹², —NR¹¹C(O)₂R¹², —NR¹¹C(O)NR¹²R¹³, —S(O)ₙR¹², —CN, —C(O)R¹², —C(O)NR¹¹R¹², C₁-C₆ alkyl, C₂-C₆ alkenyl, C₂-C₆ alkynyl, C₃-C₆ cycloalkyl or C₄-C₇ cycloalkylalkyl, and wherein each of C₁-C₆ alkyl, C₂-C₆ alkenyl, C₂-C₆ alkynyl, C₃-C₆ cycloalkyl and C₄-C₇ cycloalkylalkyl is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰; or R⁵ and R⁶ may be —O—C(R¹²)₂—O—;

R⁸ is H, halogen or OR¹¹;

R⁹ and R¹⁰ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹³, phenyl or benzyl, where phenyl or benzyl is optionally substituted from 1 to 3 times with a substituent selected independently at each occurrence thereof from halogen, cyano, C₁-C₄ alkyl, C₁-C₄ haloalkyl and C₁-C₄ alkoxy;

or R⁹ and R¹⁰ are taken together with the nitrogen to which they are attached to form a piperidine, pyrrolidine, piperazine, N-methylpiperazine, morpholine or thiomorpholine ring;

R¹¹ is H, C₁-C₄ alkyl, C₁-C₄ haloalkyl, C₁-C₄ alkoxyalkyl, C₃-C₆ cycloalkyl, C₄₋C₇ cycloalkylalkyl, —C(O)R¹³, phenyl or benzyl, where phenyl or benzyl is optionally substituted 1 to 3 times with halogen, cyano, C₁-C₄ alkyl, C₁-C₄ haloalkyl, or C₁-C₄ alkoxy;

R¹² is H, C₁-C₄ alkyl, C₁-C₄ haloalkyl, C₁-C₄ alkoxyalkyl, C₃-C₆ cycloalkyl, C₄₋C₇ cycloalkylalkyl, phenyl or benzyl, where phenyl or benzyl is optionally substituted 1 to 3 times with halogen, cyano, C₁-C₄ alkyl, C₁-C₄ haloalkyl, or C₁-C₄ alkoxy;

or R¹¹ and R¹² are taken together with the nitrogen to which they are attached to form a piperidine, pyrrolidine, piperazine, N-methylpiperazine, morpholine or thiomorpholine ring, with the proviso that only one of R⁹ and R¹⁰ or R¹¹ and R¹² are taken together with the nitrogen to which they are attached to form a piperidine, pyrrolidine, piperazine, N-methylpiperazine, morpholine or thiomorpholine ring;

R¹³ is C₁-C₄ alkyl, C₁-C₄ haloalkyl or phenyl;

n is 0, 1, or 2; and,

R¹⁴ is independently selected at each occurrence from a substituent selected from the group: halogen, —NO₂, —OR¹¹, —NR¹¹R¹², —NR¹¹C(O)R¹², —NR¹¹C(O)₂R¹², —NR¹¹C(O)NR¹²R¹³, —S(O)ₙR¹², —CN, —C(O)R¹², —C(O)NR¹¹R¹², C₁-C₆ alky, C₂-C₆ alkenyl, C₂-C₆ alkynyl, C₃-C₆ cycloalkyl, and C₄-C₇ cycloakylalkyl where C₁-C₆ alkyl, C₂-C₆ alkenyl, C₂-C₆ alkynyl, C₃-C₆ cycloalkyl, C₄-C₇ cycloalkylalkyl are optionally substituted with 1 to 3 substituents independently selected at each occurrence from the group consisting of C₁-C₃ alkyl, halogen, Ar, —CN, —OR⁹, and —NR⁹R¹⁰, or

an oxide thereof, a pharmaceutically acceptable salt thereof, a solvate thereof, or prodrug thereof

### DETAILED DESCRIPTION OF THE INVENTION

As used above, and throughout the description of the invention, the following terms, unless otherwise indicated, shall be understood to have the following meanings:

The term “Alkyl” means an aliphatic hydrocarbon group that may be straight or branched having about 1 to about 6 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, and 3-pentyl.

The term “Alkenyl” means an aliphatic hydrocarbon group containing a carbon-carbon double bond and which may be straight or branched having about 2 to about 6 carbon atoms in the chain. Preferred alkenyl groups have 2 to about 4 carbon atoms in the chain. Branched means that one or more lower alkyl groups such as methyl, ethyl or propyl are attached to a linear alkenyl chain. Exemplary alkenyl groups include ethenyl, propenyl, n-butenyl, and butenyl.

The term “Alkynyl” means an aliphatic hydrocarbon group containing a carbon-carbon triple bond and which may be straight or branched having about 2 to about 6 carbon atoms in the chain. Preferred alkynyl groups have 2 to about 4 carbon atoms in the chain. Branched means that one or more lower alkyl groups such as methyl, ethyl or propyl are attached to a linear alkynyl chain. Exemplary alkynyl groups include ethynyl, propynyl, n-butynyl, 2-butynyl, 3-methylbutynyl, and n-pentynyl.

The term “Aryl” means an aromatic monocyclic or multicyclic ring system of 6 to about 14 carbon atoms, preferably of 6 to about 10 carbon atoms. Representative aryl groups include phenyl and naphthyl.

The term “Heteroaryl” means an aromatic monocyclic or multicyclic ring system of about 5 to about 14 ring atoms, preferably about 5 to about 10 ring atoms, in which one or more of the atoms in the ring system is/are element(s) other than carbon, for example, nitrogen, oxygen or sulfur. Preferred heteroaryls contain about 5 to 6 ring atoms. The prefix aza, oxa or thia before heteroaryl means that at least a nitrogen, oxygen or sulfur atom, respectively, is present as a ring atom. A nitrogen atom of a heteroaryl is optionally oxidized to the corresponding N-oxide. Representative heteroaryls include pyrazinyl; furanyl; thienyl; pyridyl; pyrimidinyl; isoxazolyl; isothiazolyl; oxazolyl; thiazolyl; pyrazolyl; furazanyl; pyrrolyl; pyrazolyl; triazolyl; 1,2,4-thiadiazolyl; pyrazinyl; pyridazinyl; quinoxalinyl; phthalazinyl; 1(2H)-phthalazinonyl; imidazo[1,2-a]pyridine; imidazo[2,1-b]thiazolyl; benzofurazanyl; indolyl; azandolyl; benzimidazolyl; benzothienyl; quinolinyl; imidazolyl; thienopyridyl; quinazolinyl; thienopyrimidyl; pyrrolopyridyl; imidazopyridyl; isoquinolinyl; benzoazaindolyl; azabenzimidazolyl; 1,2,4-triazinyl; benzothiazolyl and the like.

The term “Alkoxy” means an alkyl-O— group wherein the alkyl group is as herein described. Exemplary alkoxy groups include methoxy, ethoxy, n-propoxy, i-propoxy, n-butoxy and heptoxy.

The term “Compounds of the invention”, and equivalent expressions, are meant to embrace compounds of general formula (1) as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

The term “Cycloalkyl” means a non-aromatic mono- or multicyclic ring system of about 3 to about 7 carbon atoms, preferably of about 5 to about 7 carbon atoms. Exemplary monocyclic cycloalkyl include cyclopentyl, cyclohexyl, cycloheptyl, and the like.

The term “Cycloalkylalkyl” means an cycloalkyl-alkyl-group in which the cycloalkyl and alkyl are as defined herein. Exemplary cycloalkylalkyl groups include cyclopropylmethyl and cyclopentylmethyl.

The term “Halo” or “halogen” means fluoro, chloro, bromo, or iodo.

The term “Haloalkyl” means both branched and straight-chain alkyl substituted with 1 or more halogen, wherein the alkyl group is as herein described.

The term “Haloalkoxy” means a C₁-C₄, alkoxy group substituted by at least one halogen atom, wherein the alkoxy group is as herein described.

The term “Substituted” or “substitution” of an atom means that one or more hydrogen on the designated atom is replaced with a selection from the indicated group, provided that the designated atom's normal valency is not exceeded. “Unsubstituted” atoms bear all of the hydrogen atoms dictated by their valency. When a substituent is keto (i.e., ═O), then 2 hydrogens on the atom are replaced. Combinations of substituents and/or variables are permissible only if such combinations result in stable compounds; by “stable compound” or “stable structure” is meant a compound that is sufficiently robust to survive isolation to a useful degree of purity from a reaction mixture, and formulation into an efficacious therapeutic agent.

The term “Pharmaceutically acceptable salts” means the relatively non-toxic, inorganic and organic acid addition salts, and base addition salts, of compounds of the present invention. These salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulphamates, malonates, sailcylates, proplonates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinateslaurylsulphonate salts, and the like. (See, for example S. M. Berge, et al., “Pharmaceutical Salts,” J. Pharm. Sci., 66: p. 1-19 (1977) and Remington's Pharmaceutical Sciences, 17^{th} ed., Mack Publishing Company, Easton, Pa., 1985, p. 1418, which are incorporated herein by reference.) Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases that include sodium hydride, sodium hydroxide, potassium hydroxide, calcium hydroxide, aluminium hydroxide, lithium hydroxide, magnesium hydroxide, zinc hydroxide. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethyl ammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., iysine and arginine, and dicyclohexylamine, and the like.

The term “Pharmaceutically acceptable prodrugs” as used herein means those prodrugs of the compounds useful according to the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” means compounds that are rapidly transformed in vivo to yield the parent compound of the above formula, for example, by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propionyl, butyryl and the like), unsubstituted and substituted aroyl (such as benzoyl and substituted benzoyl), alkoxycarbonyl (such as ethoxycarbonyl). trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard. ed., Elsevier, 1985; Methods in Enzymology, K. Widder et al, Ed., Academic Press, 421, p.309-396, 1985; A Textbook of Drug Design and Development, Krogsgaard-Larsen and H. Bundgaard. ed., Chapter 5; “Design and Applications of Prodrugs” p. 113-19 1, 1991; Advanced Drug Delivery Reviews, H. Bundgard, 8, p.1-38, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987, which are incorporated herein by reference. Examples of prodrugs include, but are not limited to, acetate, formate and benzoate derivatives of alcohol and amine functional groups in the compounds of the invention.

### Preferred Embodiments

An embodiment of the invention is the compound of formula (1) wherein:

R¹ is C₁-C₆ alkyl;

R² is H, C₁-C₆ alkyl or C₁-C₆ haloalkyl;

R³ is H, halogen, —OR¹¹, —S(O)ₙR¹²—CN, —C(O)R¹², C₁-C₆ alkyl, C₃-C₆ cycloalkyl or C₄-C₇ cycloalkylalkyl and wherein each of C₁-C₆ alkyl, C₃-C₆ cycloalkyl and C₄-C₇ cycloalkylalkyl is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰;

R⁴ is phenyl, pyridyl, pyrimidinyl, triazinyl, triazolyl, furanyl, pyranyl, indazolyl, benzimidazolyl, quinolinyl, quinazolinyl, isoquinolinyl, thienyl, imidazolyl, thiazolyl, benzthiazolyl, purinyl, isothiazolyl, indolyl, pyrrolyl, oxazolyl, benzofuranyl, benzothienyl, benzthiazolyl, isoxazolyl, and pyrazolyl, each of which is optionally substituted with from 1 to 4 R¹⁴;

R⁵ and R⁶ and R⁷ are each independently selected from the group: H, halogen, —OR¹¹, —NR¹¹R¹², —NR¹¹C(O)R², —S(O)ₙR¹², —CN, —C(O)R¹², —C(O)NR¹¹R¹², C₁-C₆ alkyl, C₃-C₆ cycloalkyl or C₄-C₇ cycloalkylalkyl, and wherein each of C₁-C₆ alkyl, C₃-C₆ cycloalkyl and C₄-C₇ cycloalkylalkyl is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰; or R⁵ and R⁶ may be —O—C(R¹²)₂—O—; and

R¹⁴ as being independently selected at each occurrence thereof from the group: halogen, —NO₂, —OR¹¹, —NR¹¹R¹², —S(O)ₙR¹², —CN, —C(O)R¹², C₁-C₆ alkyl, C₃-C₆ cycloalkyl, and C4-C7 cycloalkylalk-yl where C₁-C₆ alkyl, C₃-C₆ cycloalkyl, C₄-C₇ cycloalkylalkyl are optionally substituted with 1 to 3 substituents independently selected at each occurrence thereof from C₁-C₃ alkyl, halogen, Ar, —CN, —OR⁹, or —NR⁹R¹⁰.

Another embodiment of the invention is the compound of formula (1) wherein:

R¹ is methyl, ethyl, propyl or isopropyl;

R² is H, C₁-C₆ alkyl or C₁-C₆ haloalkyl;

R³ is H, halogen, —OR¹¹, —S(O)₂R¹², C₁-C₆ alkyl wherein C₁-C₆ alkyl is optionally substituted with 1 to 3 substituents independently selected at each occurrence thereof from C₁-C₃ alkyl, halogen, Ar, —CN, —OR⁹, or —NR⁹R¹⁰;

R⁴ is pyridyl, pyrimidinyl, triazinyl, triazolyl, furanyl, pyranyl, indazolyl, thienyl, imidazolyl, thiazolyl, puninyl, isothiazolyl, indolyl, pyrrolyl, oxazolyl, isoxazolyl, or pyrazolyl, each of which is optionally substituted with from 1 to 4 R¹⁴; and

R⁵, R⁶ and R⁷ are each independently selected from the group: H, halogen, —OR¹¹, —S(O)₂R¹², —NR¹¹R¹², —C(O)R¹², and C₁-C₆ wherein C₁-C₆ alkyl is optionally substituted with 1 to 3 substituents independently selected at each occurrence thereof from C₁-C₃ alkyl, halogen, Ar, —CN, —OR⁹, or —NR⁹R⁰.

Another embodiment of the invention is the compound of formula (1) wherein:

R₁ is CH₃;

R₂ and R₃ are each H;

R₅ and R₆ are each independently H, F Cl, OH, OCH₃ or CH₃—;

R⁷ is H or F; and

R⁸ is H, OH, or F.

Another embodiment of the invention is the compound of formula (1) wherein:

R¹ is C₁-C₆ alkyl, more preferably methyl.

Another embodiment of the invention is the compound of formula (1) wherein:

R² is H, C₁-C₆ alkyl or C₁-C₆ haloalkyl, and wherein R² is H or C₁-C₆ alkyl.

Another embodiment of the invention is the compound of formula (1) wherein R³ is H, halogen, —OR¹¹, —S(O)₂R¹², C₁-C₆ alkyl or substituted C₁-C₆ alkyl.

Another embodiment of the invention is the compound of formula (1) wherein:

R⁴ is optionally substituted aryl, or heteroaryl.

Yet another embodiment of the invention is the compound of formula (1) wherein:

R⁴ is pyridyl, pyrimidinyl, triazinyl, triazolyl, furanyl, pyranyl, indazolyl, benzimidazolyl, quinolinyl, quinazolinyl, isoquinolinyl, thienyl, imidazolyl, thiazolyl, benzthiazolyl, purinyl, isothiazolyl, indolyl, pyrrolyl, oxazolyl, benzofuranyl, benzothienyl, benzthiazolyl, isoxazolyl, pyrazolyl, oxadiazolyl, thiadiazolyl, phenyl, 2-chlorophenyl, 3-chlorophenyl, 4-chlorophenyl, 2 methoxyphenyl, 3-methoxyphenyl, 4-methoxyphenyl or 4-dimethylaminophenyl, which is optionally substituted 1-4 times with R¹⁴.

Yet another embodiment of the invention is the compound of formula (1) wherein:

R⁴ is selected from the group: 4-methyl-2-furanyl, 5-methyl-2-furanyl, 3-furanyl, 2-thienyl, 3-thienyl, 3,5-dimethyl-4-isoxazolyl, 2-pyridyl, 3-pyridyl, 4-pyridyl, 2-methoxy-3pyridyl, 6-methoxy-3-pyridyl, 3,5-pyrimidinyl and 2,6-pyrimidinyl.

Another embodiment of the invention is the compound of formula (1) wherein:

R⁵, R⁶ and R⁷ are each independently selected from the group: H, halogen, —OR¹¹, —NR¹¹R¹², —S(O)₂R¹², —C(O)R¹², and optionally substituted C₁-C₆ alkyl.

Another embodiment of the invention is the compound of formula (1) wherein:

R⁷ is H.

Another embodiment of the invention is the compound of formula (1) wherein:

R⁵ and R⁶ are each independently selected from the group: H, F, Cl, OH, OCH₃ and CH₃—.

Another embodiment of the invention is the compound of formula (1) wherein R⁸ is H, OH, or F.

Another embodiment of the invention is the compound of formula (1) wherein:

R¹ is C₁-C₆ alkyl;

R² is H, C₁-C₆ alkyl or C₁-C₆ haloalkyl;

R³ is H, halogen, —OR¹¹, —S(O)₂R¹², C₁-C₆ alkyl or substituted C₁-C₆ alkyl;

R⁴ is aryl or heteroaryl; and

R⁵, R⁶ and R⁷ are each independently H, halogen, —OR¹¹, —NR¹¹R¹², —S(O)₂R¹², C(O)R¹², C₁-C₆ alkyl or substituted C₁-C₆ alkyl.

Another embodiment of the invention is the compound of formula (1) wherein:

R¹ is methyl;

R² is H;

R³ is H;

R⁵ and R⁶ are each independently H, F, Cl, OH, OMe, or Me;

R⁷ is H or F;

R⁸ is H, OH, or F; and

R⁴ is phenyl, pyridyl, pyrimidinyl, triazinyl, triazolyl, furanyl, pyranyl, indazolyl, thienyl, imidazolyl, thiazolyl, purinyl, isothiazolyl, indolyl, pyrrolyl, oxazolyl, isoxazolyl, or pyrazolyl, each of which is optionally and independently substituted from 1-4 times with R¹⁴.

Yet another embodiment of the invention is the compound of formula (1) wherein:

R¹ is methyl;

R² is H;

R³ is H;

R⁵ and R⁶ are each H, F or CH₃;

R⁷ is H;

R⁸ is H; and

R⁴ is phenyl, 2-chlorophenyl, 3-chlorophenyl, 4-chlorophenyl, 2-methoxyphenyl, 3-methoxyphenyl, 4-methoxyphenyl, 4-dimethylaminophenyl, 4-methyl-2-furanyl, 5-methyl-2-furanyl and 3-furanyl, 2-thienyl and 3-thienyl, isoxazolyl which is 3,5-dimethyl-4-isoxazolyl, 2-pyridyl, 3-pyridyl, 4-pyridyl, 2-methoxy-3-pyridyl and 6-methoxy-3-pyridyl or 3,5-pyrimidinyl or 2,6-pyrimidinyl.

Another embodiment of the invention is the compound of formula (1) wherein the carbon atom designated * is in the R configuration.

Another embodiment of the invention is the compound of formula (1) wherein the carbon atom designated * is in the S configuration.

Another embodiment of the invention is a mixture of stereoisomeric compounds of formula (1) wherein * is in the S or R configuration.

Within these embodiments, the selection of a particular substituent at any one of R¹-R⁸ does not affect the selection of a substituent at any of the others of R¹-R⁸. That is, compounds provided herein have any of the substituents at any of the positions. For example, as described hereinabove, R¹ is can be C₁-C₆ alkyl; the selection of R¹ as any one of C₁, C₂, C₃, C₄, C₅ or C₆ alkyl, does not limit the choice of R² in particular to any one of H, C₁-C₆ alkyl or C₁-C₆ haloalkyl. Rather, for R¹ as any of C₁, C₂, C₃, C₄, C₅ or C₆ alkyl, R² is any of C₁, C₂, C₃, C₄, C₅ or C₆ alkyl or C₁, C₂, C₃, C₄, C₅ or C₆ haloalkyl. Similarly, the selection of R² as any of C₁, C₂, C₃, C₄, C₅ or C₆ alkyl or C₁, C₂, C₃, C₄, C₅ or C₆ haloalkyl does not limit the selection of R³ in particular to any one of H, halogen, —OR¹¹, —S(O)ₙR¹², —CN, —C(O)R¹², C₁-C₆ alkyl, C₃-C₆ cycloalkyl, C₄-C₇ cycloalkylalkyl or 5 substituted C₄-C₇ cycloalkylalkyl.

Other compounds of the invention are those with the following substituents:

TABLE A

| | |
|---|---|
| | (I) |
| 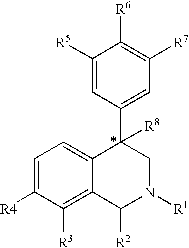 | |

| R¹ | R² | R³ | R⁴ | R⁵ | R⁶ | R⁷ | R⁸ |
|---|---|---|---|---|---|---|---|
| Me | H | H | phenyl | H | H | H | H |
| Me | H | H | 2-chlorophenyl | H | H | H | H |
| Me | H | H | 3-chlorophenyl | H | H | H | H |
| Me | H | H | 4-chlorophenyl | H | H | H | H |
| Me | H | H | 2-methoxyphenyl | H | H | H | H |
| Me | H | H | 3-methoxyphenyl | H | H | H | H |
| Me | H | H | 4-methoxyphenyl | H | H | H | H |
| Me | H | H | 4-dimethylaminophenyl | H | H | H | H |
| Me | H | H | 4-methyl-2-faranyl | H | H | H | H |
| Me | H | H | 5-methyl-2-furanyl | H | H | H | H |
| Me | H | H | 3-furanyl | H | H | H | H |
| Me | H | H | 2-thienyl | H | H | H | H |
| Me | H | H | 3-thienyl | H | H | H | H |
| Me | H | H | 3,5-dimethyl-4-isoxazole | H | H | H | H |
| Me | H | H | 2-pyridyl | H | H | H | H |
| Me | H | H | 3-pyridyl | H | H | H | H |
| Me | H | H | 4-pyridyl | H | H | H | H |
| Me | H | H | 3-pyridyl | F | F | H | H |
| Me | H | H | 2-methoxy-3-pyridyl | H | H | H | H |
| Me | H | H | 6-methoxy-3-pyridyl | H | H | H | H |
| Me | H | H | 3,5-pyrimidinyl | H | H | H | H |
| Me | H | H | 3,5-pyrimidinyl | F | F | H | H |
| Me | H | H | 3,5-pyrimidinyl | H | Me | H | H |
| Me | H | H | 2,6-pyrimidinyl | H | H | H | H |
| Me | H | H | 3,5-dimethyl-4-isoxazole | H | OMe | H | H |
| Me | H | H | 2-pyridyl | H | Ome | H | H |

wherein the carbon atom designated * is in the R or S configuration.

That is, the specific compounds provided herein include:

4,7-diphenyl-2-methyl-1,2,3,4-tetrahydroisoquinoline;

7-(2-chloro)phenyl-2-methyl-4-phenyl-1,2,3,4-tetrahydroisoquinoline;

7-(3-chloro)phenyl-2-methyl-4-phenyl-1,2,3,4-tetrahydroisoquinoline;

7-(4-chloro)phenyl-2-methyl-4-phenyl-1,2,3,4-tetrahydroilsoquinoline;

7-(2-methoxy)phenyl-2-methyl-4-phenyl-1,2,3,4-tetrahydroisoquinoline;

7-(3-methoxy)phenyl-2-methyl-4-phenyl-1,2,3,4-tetrahydroisoquinoline;

7-(4-methoxy)phenyl-2-methyl-4-phenyl-1,2,3,4-tetrahydroisoquinoline;

7-(4-N,N-dimethylamino)phenyl-2-methyl-4-phenyl-1,2,3,4-tetrahydroisoquinoline;

7-[(4-methyl)-2-thienyl-2-methyl-4-phenyl-1,2,3,4-tetrahydroisoquinoline;

7-[(5-methyl)-2-furanyl-2-methyl-4-phenyl-1,2,3,4-tetrahydroilsoquinoline;

7-(3-furanyl)-2-methyl-4-phenyl-1,2,3,4-tetrahydroisoquinoline;

2-methyl-4-phenyl-7-(2-thienyl)-1,2,3,4-tetrahydroisoquinoline;

2-methyl-4-phenyl-7-(3-thienyl)-1,2,3,4-tetrahydroisoquinoline;

7-[(3,5-dimethyl)-4-isoxazole]-2-methyl-4-phenyl-1,2,3,4-tetrahydroisoquinoline;

2-methyl-4-phenyl-7-(2-pyridyl)-1,2,3,4-tetrahydroisoquinoline;

2-methyl-4-phenyl-7-(3-pyridyl)-1,2,3,4-tetrahydroisoquinoline;

2-methyl-4-phenyl-7-(4-pyridyl)-1,2,3,4-tetrahydroisoquinoline;

4-(3,4-difluoro)phenyl-2-methyl-7-(3-pyridyl)-1,2,3,4-tetrahydroisoquinoline;

7-[(2-methoxy)-3-pyridyl-2-methyl-4-phenyl-1,2,3,4-tetrahydroisoquinoline;

7-[(6-methoxy)-3-pyridyl-2-methyl-4-phenyl-1,2,3,4-tetrahydroisoquinoline;

2-methyl-4-phenyl-7-(3,5-pyrimidyl)-1,2,3,4-tetrahydrolsoquinoline;

4-(3,4-difluoro)phenyl-2-methyl-7-(3,5-pyrimidyl)-1,2,3,4-tetrahydrolsoquinoline;

4-(4-methyl)phenyl-2-methyl-7-(3,5-pyrimidyl)-1,2,3,4-tetrahydroisoquinoline;

2-methyl-4-phenyl-7-(2,6-pyrimidyl)-1,2,3,4-tetrahydroisoquinoline;

7-(2,5-dimethyl-4-isoxazole)-4-(4-methoxy)phenyl-2-methyl-1,2,3,4-tetrahydroisoquinoline; and

4-(4-methoxy)phenyl-2-methyl-7-(2-pyridyl)-1,2,3,4-tetrahydroisoquinoline or an oxide thereof, a pharmaceutically acceptable salt thereof, a solvate thereof, or a prodrug thereof.

Another aspect of the invention is a mixture of compounds of formula (1) wherein the compound of formula (1) is radiolabeled, i.e., wherein one or more of the atoms described are replaced by a radioactive isotope of that atom (e.g., C replaced by “C and H 3 replaced by H). Such compounds have a variety of potential uses, e.g., as standards and reagents in determining the ability of a potential pharmaceutical to bind to neurotransmitter proteins.

“Therapeutically effective amounts” are any amounts of the compounds effective to ameliorate, lessen, inhibit or prevent the particular condition for which a subject is being treated. Such amounts generally vary according to a number of factors well within the purview of ordinarily skilled artisans given the description provided herein to determine and account for. These include, without limitation: the particular subject, as well as its age, weight, height, general physical condition and medical history; the particular compound used, as well as the carrier in which it is formulated and the route of administration selected for it; and, the nature and severity of the condition being treated. Therapeutically effective amounts include optimal and suboptimal doses, and can be determined in a variety of ways known to ordinarily skilled artisans, e.g., by administering various amounts of a particular agent to an animal afflicted with a particular condition and then determining the relative therapeutic benefit received by the animal. The amounts generally range from about 0.001 mg per kg of the body weight of the subject being treated to about 1000 mg per kg, and more typically, from about 0.1 to about 200 mg per kg. These amounts can be administered according to any dosing regimen acceptable to ordinarily skilled artisans supervising the treatment. More specific doses are mentioned below in relationship to the treatment of particular disorders that are the subject of this invention.

“Pharmaceutically acceptable carriers”, are media generally accepted in the art for the administration of therapeutic compounds to humans. Such carriers are generally formulated according to a number of factors well within the purview of those of ordinary skill in the art to determine and account for. These include, without limitation: the type and nature of the active agent being formulated; the subject to which the agent-containing composition is to be administered; the intended route of administration of the composition; and, the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, well known to those of ordinary skill in the art. Descriptions of suitable pharmaceutically acceptable carriers, and factors involved in their selection, are found in a variety of readily available sources, e.g., Remington's Pharmaceutical Sciences, 17^{th} ed., Mack Publishing Company, Easton, Pa., 1985, the contents of which are incorporated herein by reference.

Compounds of this invention are administered, for example, parenterally in various aqueous media such as aqueous dextrose and saline solutions; glycol solutions are also useful carriers. Solutions for parenteral administration preferably contain a water soluble salt of the active ingredient, suitable stabilizing agents, and if necessary, buffer substances. Antioxidizing agents, such as sodium bisulfite, sodium sulfite, or ascorbic acid, either alone or in combination, are suitable stabilizing agents. Also used are citric acid and its salts, and EDTA. In addition, parenteral solutions can contain preservatives such as benzalkonium chloride, methyl- or propylparaben, and chlorobutanol.

Alternatively, the compounds are administered orally in solid dosage forms, such as capsules, tablets and powders; or in liquid forms such as elixirs, syrups, and/or suspensions. Gelatin capsules can be used to contain the active ingredient and a suitable carrier such as but not limited to lactose, starch, magnesium stearate, steric acid, or cellulose derivatives. Similar diluents can be used to make compressed tablets. Both tablets and capsules can be manufactured as sustained release products, to provide for continuous release of medication over a period of time. Compressed tablets can be sugar-coated or film-coated to mask any unpleasant taste, or used to protect the active ingredients from the atmosphere, or to allow selective disintegration of the tablet in the gastrointestinal tract.

Compounds of this invention provide a particularly beneficial therapeutic index relative to other compounds available for the treatment of similar disorders. Without intending to be limited by theory, it is believed that this is due, at least in part, to the compounds, ability to be selective for the norepinephrine transporter protein (NET) over the other neurotransmitter transporters. Binding affinities are demonstrated by a number of means well known to ordinarily skilled artisans.

Briefly, for example, protein-containing extracts from cells, e.g., HEK293 cells, expressing the transporter proteins are incubated with radiolabeled ligands for the proteins. The binding of the radioligands to the proteins is reversible in the presence of other protein ligands, e.g., the compounds of this invention; said reversibility, as described below, provides a means of measuring the compounds' binding affinities for the proteins (Ki). A higher Ki value f or a compound is indicative that the compound has less binding affinity for a protein than is so for a compound with a lower Ki; conversely, lower Ki values are indicative of greater binding affinities.

Accordingly, a lower Ki for the protein for which the compound is more selective, and a higher Ki for the protein for which the compound is less selective indicate the difference in compound selectivity for proteins. Thus, the higher the ratio in Ki values of a compound for protein A over protein B, the greater is the compounds' selectivity for the latter over the former (the former having a higher Ki and the latter a lower Ki for that compound). Compounds provided herein induce f ewer side effects during therapeutic usage because of their selectivity for the norepinephrine transporter protein, as indicated by the ratios of their Ki's for binding to NET over those for binding to other transporter proteins, e.g., the dopamine transporter (DAT) and the serotonin transporter (SERT). Generally, the compounds of this invention have a Ki ratio for DAT/NET of about ≧2:1; the compounds generally also have a SERT/NET ratio of about ≧5:1.

Moreover, in vivo assessment of the activity of compounds at the NE and DA transporters is, for example, by determining their ability to prevent the sedative effects of tetrabenazine (TBZ) (see, e.g., G. Stille, Arzn. Forsch. 1964, 14, 534-537; the contents of which are incorporated herein by reference). Randomized and coded doses of test compounds are administered to mice, as is then a dose of tetrabenazine. Animals are then evaluated for antagonism of tetrabenazine-induced exploratory loss and ptosis at specified time intervals after drug administration. Exploratory activity is, for example, evaluated by placing the animal in the center of a circle and then evaluating the amount of time it takes for the animal to intersect the circle's perimeter—generally, the longer it takes for the animal to make this intersection, the greater is its loss of exploratory activity. Furthermore, an animal is considered to have ptosis if its eyelids are at least 50% closed. Greater than 95% of the control (vehicle-treated) mice are expected to exhibit exploratory loss and ptosis; compound-related activity is then calculated as the percentage of mice failing to respond to the tetrabenazine challenge dose, with therapeutically more effective compounds expected to be better at reducing loss of exploratory behavior and ptosis.

Accordingly, the pharmaceutical compositions provided herein are useful in the treatment of subjects afflicted with various neurological and psychiatric disorders by administering to said subjects a dose of a pharmaceutical composition provided herein. Said disorders include, without limitation, chronic and neuropathic pain, migraine therapy and prevention, and urge, stress and mixed urinary incontinence. The compounds provided herein, are particularly useful in the treatment of these and other disorders due, at least in part, to their ability to selectively bind to the transporter proteins for certain neurochemicals with a greater affinity than to the transporter proteins for other neurochemicals.

The compounds of the present invention can be prepared using the methods described in International Application WO 01/32625, together with methods known in the art of synthetic organic chemistry, or variations thereof as appreciated by those skilled in the art.

In order to evaluate the relative affinity of the various compounds at the NE, DA and 5HT transporters, HEK293E cell lines can be developed to express each of the three human transporters. cDNAs containing the complete coding regions of each transporter can be amplified by PCR from human brain libraries. The cDNAs contained in pCRII vectors can be sequenced to verify their identity and then subcloned into an Epstein Barr virus based expression plasmid (E. Shen, G M Cooke, R A Horlick, Gene 156:235-239, 1995). This plasmid containing the coding sequence for one of the human transporters can be transfected into HEK293E cells. Successful transfection can be verified by the ability of known reuptake blockers to inhibit the uptake of tritiated NE, DA or 5HT.

For binding, cells can be homogenized, centrifuged and then resuspended in incubation buffer (50 mM Tris, 120 mM NaCl, 5 mM KCl, pH 7.4). Then the appropriate radioligand can be added. For NET binding, [³H] Nisoxetine (86.0 Ci/mmol, NEN/DuPont) can be added to a final concentration of approximately 5 nM. For DAT binding, [³H] WIN 35,428 (84.5 Ci/mmol) at 15 nM was added. For 5HTT binding, [³H] Citolapram (85.0 Ci/mmol) at 1 nM was added. Then various concentrations (10---5 to IOA-11 M) of the compound of interest can be added to displace the radioligand. Incubation can be carried out at room temperature for 1 hour in a 96 well plate. Following incubation, the plates can be placed on a harvester and washed quickly 4 times with (50 mM tris, 0.9% NaCl, pH 7.4) where the cell membranes containing the bound radioactive label can be trapped on Whatman GF/B filters. Scintillation cocktail can be added to the filters which were then counted in a Packard TopCount. Binding affinities of the compounds of interest can be determined by non-linear curve regression using GraphPad Prism 2.01 software. Non-specific binding can be determined by displacement with 10 micromolar mazindol.

In order to assess in vivo activity of the compounds at the NE and DA transporters, their ability to prevent the sedative effects of tetrabenazine (TBZ) can be determined (G. Stille, Arzn. Forsch 14:534-537, 1964). Male CFI mice (Charles River Breeding Laboratories) weighing 18-25 gm at the time of testing, can be housed a minimum of 6 days under carefully controlled environmental conditions (22.2+1.1 C; 50% average humidity; 12 hr lighting cycle/24 hr). Mice can be fasted overnight (16-22 hr) prior to testing. Mice can be placed into clear polycarbonated “shoe” boxes (17 cm×28.5 cm×12 cm).

Randomized and coded doses of test compounds can be administered p.o. A 45 mg/kg dose of tetrabenazine can be administered i.p. 30 minutes prior to score time. All compounds can be administered in a volume of 0.1 ml/10 gm body weight. Animals can be evaluated for antagonism of tetrabenazine induced exploratory loss and ptosis at specified time intervals after drug administration. At the designated time interval, mice are examined for signs of exploratory activity and ptosis. Exploratory activity can be evaluated by placing the animal in the center of a 5-inch circle. Fifteen seconds can be allowed for the animal to move and intersect the perimeter. This can be considered antagonism of tetrabenazine and given a score of 0. Failure to leave the circle can be regarded as exploratory loss and given a score of 4. An animal can be considered to have ptosis if its eyelids are at least 50% closed and can be given a score of 4 if completely closed; no closure can be given a score of 0. Greater than 95% of the control (vehicle-treated) mice can be expected to exhibit exploratory loss and ptosis. Drug activity can be calculated as the percentage of mice failing to respond to the tetrabenazine challenge dose.

Median effective doses (ED50s) and 95% confidence limits 30 can be determined numerically by the methods of Thompson (1947) and Litchfield and Wilcoxon (1949).

Chronic painful conditions, in various forms, affect a considerable number of people including, according to the WHO, 4 million cancer sufferers who, worldwide, suffer as a result of a lack of suitable care. There are a number of other conditions, such as musculoskeletal or vertebral pain, neurological pain, headaches or vascular pain. Neurophathic pain, a chronic pain condition occurring in the setting of nervous system injury or tissue injury, is characterized by unusual sensory experiences (allodynia, hyperalgesia) and abnormal pain processing in the central and peripheral nervous systems; treatment of neuropathic pain is difficult. Painful diabetic neuropathy is one of the most frequent complications of diabetes in humans, post-herpetic neuralgia develops in 10-30% of patients after herpes zoster, phantom limb and stump pain is a common sequela of amputation. Chronic pain may also be caused by a trauma, an entrapment neuropathy (e.g. carpal tunnel syndrome), multiple sclerosis or a polyneurophathy associated with AIDS, alcoholism, hypothyroidism, or anticancer chemotherapy.

Conventional treatments of pain fall into two categories: 1) nonsteroidal anti-inflammatory drugs (NSAIDs), used to treat mild pain, but whose therapeutic use is limited by GI adverse effects; and 2) morphine and related opiods, used to treat moderate to severe pain but whose therapeutic use is limited by undesirable side effects including respiratory depression, tolerance, and abuse potential. However, conventional analgesics, whether opiates or NSAIDs, have limited therapeutic value in the management of chronic pain syndromes. This has led to the use of adjuvant analgesics for the management of these conditions. For example, tricyclic antidepressant are currently the first choice in the treatment of painful diabetic neuropathy. However, few agents are fully effective in all patients and undesirable side effects are common.

For use in the treatment of chronic pain or neuropathic pain the compounds of formula IA, IB, IIA, IIB, IIIA, and IIIB may be administered orally or parenterally in an amount sufficient to alleviate the symptoms of chronic pain or neuropathic pain. The actual amount of a compound of formula I to be used will vary with the severity and nature of the state of chronic or neuropathic pain, the animal being treated and the level of relief sought. In the human, an oral dose of from about 2 to about 50 milligrams, administered as needed represents appropriate posology. Intramuscular administration of from about 1 to about 25 milligrams provides a dosage comparable to that specified for oral administration.

As used herein the term “chronic pain” means pain selected from causalgia, neuropathic pain, diabetic neuropathy, post-surgery or traumatic neuropathy, postherpetic neuralgia, peripheral neuropathy, entrapment neuropathy, phantom limb and stump pain, neuropathy caused by alcohol abuse, HIV infection, multiple sclerosis hypothyroidism, lower back pain, cancer pain and pain from anticancer chemotherapy. Applicant particularly prefers the use of the compounds of formula IA, IB, IIA, IIB, IIIA, and IIIB for the treatment of neuropathic pain.

The “term chronic pain relieving amount” represents an amount of a compound of formula IA, IB, IIA, IIB, IIIA, and IIIB which is capable of relieving or reducing chronic pain in a mammal in need thereof. The pain of migraine is associated with excessive dilatation of the cranial vasculature and known treatments for migraine include the administration of compounds having vasoconstrictor properties such as ergotamine. However, ergotamine is a non-selective vasoconstrictor that constricts blood vessels throughout the body and has undesirable and potentially dangerous side effects. Migraine may also be treated by administering an analgesic, usually in combination with an antiemetic, but such treatments are of limited value.

There is thus a need for a safe and effective drug for the treatment of migraine, which can be used either prophylactically or to alleviate an established headache, and a compound having a selective vasoconstrictor activity would fulfill such a role.

Furthermore, in conditions such as migraine, where the drug will usually be administered by the patient, it is highly desirable that the drug can be taken orally. It should therefore possess good bioavailability and be effectively absorbed from the gastro-intestinal tract so that prompt relief of symptoms can occur. The drug should also be safe (i.e., free from toxic effects) when administered by the oral route.

It is generally believed that the pain of migraine is of vascular origin and caused by excessive dilation of branches of the common carotid arterial bed. (J. W. Lance, Mechanisms and Management of Migraine, Butterworths, p 113-152 (1973). The role of norepinephrine reuptake in the management of migraine headache pain is discussed in J. R. Couch, et al., Amitriptyline in the prophylaxis of migraine, Neurology 1976:26:121-127 and S. Diamond, et al., Chronic tension headache treated with amitruptyline: a double blind study, Headache 1971; 11:110-116.

A proposed dose of the compounds of the invention for oral administration to man (about 70 kg bodyweight) for the treatment of migraine is 0.1 mg to 100 mg, for example 0.5 mg to 50 mg, preferably 2 mg to 40 mg, of the active ingredient per dose which could be administered up to 4 times per day, more usually 1 to 2 times per day. It will be appreciated that it may be necessary to make routine variations to the dosage depending on the age and weight of the patient, as well as the severity of the condition to be treated. It should be understood that unless otherwise indicated, the dosages are referred to in terms of the weight of compound (I) as the free base.

According to a further aspect, the invention provides a method of treatment of a human subject suffering from or susceptible to pain resulting from dilatation of the cranial vasculature, such as migraine or cluster headache, by administration of a compound of formula (I) or a physiologically acceptable salt or solvate thereof. The method of treatment preferably comprises oral administration of a compound of the invention.

Urinary incontinence is generally defined as the involuntary loss of urine and is most common in four groups of patients including children, women, elderly, and neurologic disease patients. Detrusor instability is characterized by spasmodic bladder contractions or bladder contractions elicited by small volumes, and is often accompanied by incontinence and urinary frequency. Interstitial cystitis is an idiopathic pelvic pain syndrome that can also include detrusor instability as a component of its pathology.

Nocturnal enuresis is classified as an involuntary micturition during sleep after 5 years of age and may exist in either primary or secondary forms. The diagnosis of primary nocturnal enuresis is made if the patient has never developed voluntary control of micturition during sleep. The diagnosis of secondary nocturnal enuresis is made if the patient has had transient periods of micturition control during sleep. Nocturnal enuresis occurs in 30% of all children at 4 years of age, 10% at 6 years, 3% at 10 years and 1% at 18 years. Secondary nocturnal enuresis accounts for approximately 20-25% of the pediatric enurenic cases. Although some enuretic children also have diurnal enuresis, over 80% of the enuretic children have exclusively nocturnal enuresis.

The predominant types of incontinence in women are stress and urge incontinence. Stress incontinence is the involuntary loss of urine through an intact urethra produced during times of increased abdominal pressure such as during physical activity and coughing. This implies that the urethra cannot generate sufficient pressure for outlet resistance to compensate for increases in intrabladder pressure. This loss of urine is not accompanied by premonitory sensations of the need to void and is not related to the fullness of the bladder. Urge incontinence is the involuntary loss of urine through an intact urethra due to an increased intrabladder pressure. In contrast to stress incontinence, urge incontinence is caused by an episodic bladder contraction (detrusor instability) which exceeds the outlet resistance pressure generated by the urethra and is accompanied by a perception of urgency to void.

Stress incontinence is the most common form of incontinence in young women. In two longitudinal studies, pure stress incontinence was found to occur in 15-22% of women from ages 17-75+. The highest incidence of stress incontinence (25-30%) occurs at 25-45 years of age or during the childbearing years. Following the first childbirth, the overall incidence and incidence of severe stress incontinence doubles. However, 35-50% of nulliparous women have also occasional stress incontinence. In a study of nulliparous nursing students between the ages of 17-24 years, daily stress incontinence was reported in 17% of the women. Urge incontinence occurs in approximately 10% of women from ages 17-75+ years and increases progressively with age. In addition to stress or urge incontinence, 7-14% of women from ages 17-75+ years of age have characteristics of both urge and stress incontinence. The incidence of this “complex incontinence” doubles during the childbearing years and ranges from 13-28% from ages 17 to 75+ years of age.

The types of incontinence seen in the elderly include urge incontinence (detrusor instability), stress incontinence, complex incontinence (urge and stress incontinence) and total incontinence. Urge incontinence is the most common form of incontinence in the elderly men and women and is caused by abnormal neuromuscular responses of the bladder. Following urge incontinence in incidence are complex, stress, overflow and total incontinence, respectively. Stress incontinence is relatively rare in elderly men but common in women. Stress incontinence is caused by pelvic surgery, anatomical changes in the orientation of the bladder and urethra, decreased tone of the pelvic muscles, deterioration of the urethra following the cessation of estrogen secretion, and idiopathic decrease in the neuromuscular response of tile urethra. Overflow incontinence is due to an overfilling and distension of an areflexic bladder that exceeds the urethral resistance. Total incontinence is associated with dementia and sphincter or nerve damage.

In addition to the types of incontinence described above, urge incontinence is also associated with neurologic disorders such as multiple sclerosis, Alzheimer's disease and Parkinson's disease. This urge incontinence caused by neurologic disorders result from bladder hyperactivity. The incidence of incontinence in multiple sclerosis patients has been estimated to be 60-90%. Urinary incontinence is among the early neurologic symptoms of Parkinson's disease patients and is frequently exacerbated by treatment with anti-Parkinson drugs.

Interstitial cystitis is a syndrome that is characterized by increases in urination frequency, urgency, suprapubic pressure and pain with bladder filling. This syndrome is not associated with infections or cytological damage. The average age at onset of this disorder is 40-50 years. The quality of life is considered to be worse than that of end stage renal disease. According to the NIH report on interstitial cystitis, there are 20,000 to 90,000 diagnosed cases of this disorder in United States and the upper boundary for undiagnosed cases is 4-5 times larger than the range of diagnosed cases. This disorder has increased in awareness in the urologic community due to the formation of the American Interstitial Cystitis Association.

The treatments for incontinence vary with the particular type. For example, with no therapy, the spontaneous cure rate for nocturnal enuresis is approximately 15% per year. The success rate for nonpharmacologic therapies such as motivational counseling, bladder exercises and enuresis alarms ranges from 25-70%. The tricyclic antidepressants have been the most effective pharmacologic agents for treating nocturnal enuresis. Imipramine is the most widely used agent; however other tricyclics such as nortriptyline, amitriptyline, and desipramine are also effective. Enuresis can be cured in over 50% of patients following treatment with imipramine and improvements can be seen in another 15-20%. A successful response to this therapy is usually seen in the first week of therapy and often after the first dose. The best results are seen in children with normal sized bladders who are occasionally continent at night. The worst results are seen in children with small bladders and in older adolescents. This therapy, however, does have toxic risks. The tricyclic anti-depressants in general, and imipramine in particular, are not approved for use in children under 5 years of age as these compounds are particularly toxic and potentially lethal in low dosage. Other pharmacologic therapies include the use of oxybutynin, antispasmotic agent that reduces uninhibited detrusor muscles contractions, and the antidiuretic agent desmopressin.

The predominant forms of therapy for incontinent women include a variety of surgical procedures that attempt to resuspend the bladder and/or reinforce the urethra; pelvic floor exercises; and pharmacologic therapies. Imipramine is effective as a single therapy in restoring continence to women with stress incontinence. The efficacy of imipramine in urge incontinence has varied along clinical studies and appears greater when used as a combination therapy with anticholinergic and antispasmotic agents.

The amount of compound required to effectively treat incontinence will depend upon the compound employed and its relative potency for effecting monoamine reuptake inhibition. Such doses can be generally extrapolated based upon the in vitro and any in vivo testing such as that mentioned above. For example, for adult patients, a compound of this invention would be expected to be effective when administered in amounts of 20-200 milligrams per day. However, it should be readily understood that the amount of the compound actually administered will be determined by a physician, in light of all the relevant circumstances including the particular condition to be treated, the choice of compound to be administered, and the choice of route of administration.

What is claimed is:

1. A method of treating chronic or neuropathic pain, treating or preventing migraine headaches, or treating stress, urge or mixed urinary incontinence comprising administering to a patient in need thereof an effective amount of a compound of the formula (1): 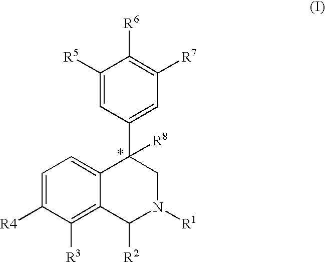
wherein:
the carbon atom designated * is in the R or S configuration;
R¹ is C₁-C₆ alkyl, C₂-C₆ alkenyl, C₂-C₆ alkynyl, C₃-C₆ cycloalkyl or C₄-C₇ cycloalkylalkyl, each of which is optionally substituted with from 1 to 3 substituents independently selected at each occurrence thereof from C₁-C₃ alkyl, halogen, Ar, —CN, —OR⁹ and —NR⁹R¹⁰;
R² is H, C₁-C₆ alkyl, C₂-C₆ alkenyl, C₂-C₆ alkynyl, C₃-C₆ cycloalkyl, C₄-C₇ cycloalkylalkyl or C₁-C₆ haloalkyl;
R³ is H, halogen, —OR¹¹, —S(O)ₙR¹², —CN, —C(O)R¹², —C(O)NR¹¹R¹², C₁-C₆ alkyl, C₂-C₆ alkenyl, C₂-C₆ alkynyl, C₃-C₆ cycloalkyl or C₄-C₇ cycloalkylalkyl and wherein each of C₁-C₆ alkyl, C₂-C₆ alkenyl, C₂-C₆ alkynyl, C₃-C₆ cycloalkyl and C₄-C₇ is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰;
R⁴ is aryl selected from phenyl, naphthyl and indenyl, or heteroaryl selected from pyridyl, pyrimidinyl, triazinyl, triazolyl, furanyl, pyranyl, indazolyl, benzimidazolyl, quinolinyl, quinazolinyl, isoquinolinyl, thienyl, imidazolyl, thiazolyl, benzthiazolyl, purinyl, isothiazolyl, indolyl, pyrrolyl, oxazolyl, benzofuranyl, benzothienyl, benzthiazolyl, isoxazolyl, pyrazolyl, oxadiazolyl and thiadiazolyl, wherein the aryl or heteroaryl group is optionally substituted with from 1 to 4 R¹⁴ substituents;
R⁵ and R⁶ and R⁷ are each independently H or are selected from halogen, —OR¹¹, —NR¹¹R¹², —NR¹¹C(O)R¹², —NR¹¹C(O)₂R¹², —NR¹¹C(O)NR¹²R¹³, —S(O)ₙR¹², —CN, —C(O)R¹², —C(O)NR¹¹R¹², C₁-C₆ alkyl, C₂-C₆ alkenyl, C₂-C₆ alkynyl, C₃-C₆ cycloalkyl or C₄-C₇ cycloalkylalkyl, and wherein each of C₁-C₆ alkyl, C₂-C₆ alkenyl, C₂-C₆ alkynyl, C₃-C₆ cycloalkyl and C₄-C₇ cycloalkylalkyl is optionally substituted with from 1 to 3 substituents independently selected at each occurrence thereof from C₁-C₃ alkyl, halogen, —CN, —OR⁹, —NR⁹R¹⁰ and phenyl which is optionally substituted 1-3 times with halogen, cyano, C₁-C₄ alkyl, C₁-C₄ haloalkyl, or C₁-C₄ alkoxy, —CN, —OR⁹, or —NR⁹R¹⁰; or R⁵ and R⁶ may be —O—C(R¹²)₂—O—;
R⁸ is H, halogen or OR¹¹;
R⁹ and R¹⁰ are each independently H, C₁-C₄ alkyl, C₁-C₄ haloalkyl, C₁-C₄ alkoxyalkyl, C₃-C₆ cycloalkyl, C₄-C₇ cycloalkylalkyl, —C(O)R¹³, phenyl or benzyl, where phenyl or benzyl is optionally substituted from 1 to 3 times with a substituent selected independently at each occurrence thereof from halogen, cyano, C₁-C₄ alkyl, C₁-C₄ haloalkyl and C₁-C₄ alkoxy;
or R⁹ and R¹⁰ are taken together with the nitrogen to which they are attached to form a piperidine, pyrrolidine, piperazine, N-methylpiperazine, morpholine or thiomorpholine ring;
R¹¹ is H, C₁-C₄ alkyl, C₁-C₄ haloalkyl, C₁-C₄ alkoxyalkyl, C₃-C₆ cycloalkyl, C₄₋C₇ cycloalkylalkyl, —C(O)R¹³, phenyl or benzyl, where phenyl or benzyl is optionally substituted 1 to 3 times with halogen, cyano, C₁-C₄ alkyl, C₁-C₄ haloalkyl, or C₁-C₄ alkoxy;
R¹² is H, C₁-C₄ alkyl, C₁-C₄ haloalkyl, C₁-C₄ alkoxyalkyl, C₃-C₆ cycloalkyl, C₄₋C₇ cycloalkylalkyl, phenyl or benzyl, where phenyl or benzyl is optionally substituted 1 to 3 times with halogen, cyano, C₁-C₄ alkyl, C₁-C₄ haloalkyl, or C₁-C₄ alkoxy;
or R¹¹ and R¹² are taken together with the nitrogen to which they are attached to form a piperidine, pyrrolidine, piperazine, N-methylpiperazine, morpholine or thiomorpholine ring, with the proviso that only one of R⁹ and R¹⁰ or R¹¹ and R¹² are taken together with the nitrogen to which they are attached to form a piperidine, pyrrolidine, piperazine, N-methylpiperazine, morpholine or thiomorpholine ring;
R¹³ is C₁-C₄ alkyl, C₁-C₄ haloalkyl or phenyl;
n is 0, 1, or 2; and,
R¹⁴ is independently selected at each occurrence from a substituent selected from the group: halogen, —NO₂, —OR¹¹, —NR¹¹R¹², —NR¹¹C(O)R¹², —NR¹¹C(O)₂R¹², NR¹¹C(O)NR¹²R, —S(O)ₙR¹², —CN, —C(O)R¹², —C(O)NR¹R¹², C—C₆ alkyl, C₂-C₆ alkenyl, C₂-C₆ alkynyl, C₃-C₆ cycloalkyl, and C₄-C₇ cycloalkylalkyl where C₁-C₆ alkyl, C₂-C₆ alkenyl, C₂-C₆ alkynyl, C₃-C₆ cycloalkyl, C₄-C₇ cycloalkylalkyl are optionally substituted with 1 to 3 substituents independently selected at each occurrence from the group consisting of C₁-C₃ alkyl, halogen, Ar, —CN, —OR⁹, and —NR⁹R¹⁰, or
an oxide thereof, a pharmaceutically acceptable salt thereof, a solvate thereof, or prodrug thereof.

2. A method of claim 1, wherein R¹ is C₁-C₆ alkyl.

3. A method of claim 2, wherein R¹ is methyl.

4. A method of claim 1, wherein R² is H, C₁-C₆ alkyl or C₁-C₆ haloalkyl.

5. The compound of claim 4, wherein R² is H or C₁-C₆ alkyl.

6. A method of claim 5, wherein R² is H.

7. A method of claim 1, wherein R³ is H, halogen, —OR¹¹, —S(O)₂R¹², C₁-C₆ alkyl or substituted C₁-C₆ alkyl.

8. A method of claim 7, wherein R³ is H.

9. A method of claim 1, wherein R⁴ is phenyl optionally and independently substituted from 1 to 4 times with R¹⁴.

10. A method of claim 9, wherein the R⁴ is phenyl, 2-chlorophenyl, 3-chlorophenyl, 4 chlorophenyl, 2-methoxyphenyl, 3-methoxyphenyl, 4-methoxyphenyl or 4 dimethylaminophenyl.

11. A method of claim 1, wherein R⁴ is pyridyl, pynmidinyl, triazinyl, triazolyl, furanyl, pyranyl, indazolyl, benzimidazolyl, quinolinyl, quinazolinyl, isoquinolinyl, thienyl, imidazolyl, thiazolyl, benzthiazolyl, puninyl, isothiazolyl, indolyl, pyrrolyl, oxazolyl, benzofuranyl, benzothienyl, benzthiazolyl, isoxazolyl, pyrazolyl, oxadiazolyl, or thiadiazolyl, which is optionally substituted 1-4 times with R¹⁴.

12. A method of claim 11, wherein R⁴ is 4-methyl-2-furanyl, 5-methyl-2-furanyl, 3-furanyl, 2-thienyl, 3-thienyl, 3,5-dimethyl-4-isoxazolyl, 2-pyridyl, 3-pyridyl, 4-pyridyl, 2-methoxy-3-pyridyl, 6-methoxy-3pyridyl, 3,5-pyrimidinyl or 2,6-pyrimidinyl.

13. The compound of claim 1, wherein R⁵, R⁶ and R⁷ are each independently selected from the group: H, halogen, —OR¹¹, —NR¹¹R¹², -, —S(O)₂R¹², —C(O)R¹² and optionally substituted C₁-C₆ alkyl.

14. A method of claim 13, wherein R⁷ is H.

15. A method of claim 14, wherein of R⁵ and R⁶ are each H, F, Cl, OH, OCH₃ or CH₃—.

16. A method of claim 1, wherein R⁸ is H, OH, or F.

17. A method of claim 1, wherein
R¹ is C₁-C₆ alkyl;
R² is H, C₁-C₆ alkyl or C₁-C₆ haloalkyl;
R³ is H, halogen, —OR¹¹, —S(O)₂R¹², C₁-C₆ alkyl or substituted C₁-C₆ alkyl;
R⁴ is aryl or heteroaryl; and
R⁵, R⁶ and R⁷ are each independently selected from the group: H, halogen, —OR¹¹, NR¹¹R¹², —S(O)₂R¹², —C(O)R¹², C₁-C₆ alkyl and substituted C₁-C₆ alkyl.

18. A method of claim 1, wherein R¹ is methyl;
R² is H;
R³ is H;
R⁵ and R⁶ are each independently selected from the group: H, F, Cl, OH, OCH₃, and CH₃;
R⁷ is H or F;
R⁸ is H, OH, or F; and
R⁴ is phenyl, pyridyl, pyrimidinyl, triazinyl, triazolyl, furanyl, pyranyl, indazolyl, thienyl, imidazolyl, thiazolyl, purinyl, isothiazolyl, indolyl, pyrrolyl, oxazolyl, isoxazolyl, or pyrazolyl, each of which R⁴ is optionally and independently substituted from 1-4 times with R¹⁴.

19. A method of claim 1, wherein R¹ is methyl;
R² is H;
R³ is H;
R⁵ and R⁶ are each H, F or CH₃;
R⁷ is H;
R⁸ is H; and
R⁴ is phenyl, 2-chlorophenyl, 3-chlorophenyl, 4-chlorophenyl, 2-methoxyphenyl, 3 methoxyphenyl, 4-methoxyphenyl, 4-dimethylaminophenyl, 4-methyl-2-furanyl, 5 methyl-2-furanyl and 3-furanyl, 2-thienyl and 3-thienyl, 3,5-dimethyl-4-isoxazolyl, 2 pyridyl, 3-pyridyl, 4-pyridyl, 2-methoxy-3-pyridyl and 6-methoxy-3-pyridyl 3,5 pyrimidinyl or 2,6-pyrimidinyl.

20. A method according to claim 1, wherein the carbon atom designated * is in the R configuration.

21. A method according to claim 1, wherein the carbon atom designated * is in the S configuration.

22. A method comprising a mixture of stereoisomerisms compounds of claim 1 wherein the carbon atom designated * is in the S or R configuration.

23. A method according to claim 1, selected from the group:
4,7-diphenyl-2-methyl-1,2,3,4-tetrahydroisoquinoline;
7-(2-chloro)phenyl-2-methyl-4-phenyl-1,2,3,4-tetrahydroisoquinoline;
7-(3-chloro)phenyl-2-methyl-4-phenyl-1,2,3,4-tetrahydroisoquinoline;
7-(4-chloro)phenyl-2-methyl-4-phenyl-1,2,3,4-tetrahydroisoquinoline;
7-(2-methoxy)phenyl-2-methyl-4-phenyl-1,2,3,4-tetrahydroisoquinoline;
7-(3-methoxy)phenyl-2-methyl-4-phenyl-1,2,3,4-tetrahydroisoquinoline;
7-(4-methoxy)phenyl-2-methyl-4-phenyl-1,2,3,4-tetrahydroisoquinoline;
7-(4-N,N-dimethylamino)phenyl-2-methyl-4-phenyl-1,2,3,4-tetrahydroisoquinoline;
7-[(4-methyl)-2-thienyl-2-methyl-4-phenyl-1,2,3,4-tetrahydroisoquinoline;
7-[(5-methyl)-2-furanyl]-2-methyl-4-phenyl-1,2,3,4-tetrahydroisoquinoline;
7-(3-furanyl)-2-methyl-4-phenyl-1,2,3,4-tetrahydroisoquinoline;
2-methyl-4-phenyl-7-(2-thienyl)-1,2,3,4-tetrahydroisoquinoline;
2-methyl-4-phenyl-7-(3-thienyl)-1,2,3,4-tetrahydroisoquinoline;
7-[(3,5-dimethyl)-4-isoxazole]-2-methyl-4-phenyl-1,2,3,4-tetrahydroisoquinoline;
2-methyl-4-phenyl-7-(2-pyridyl)-1,2,3,4-tetrahydroisoquinoline;
2-methyl-4-phenyl-7-(3-pyridyl)-1,2,3,4-tetrahydroisoquinoline;
2-methyl-4-phenyl-7-(4-pyridyl)-1,2,3,4-tetrahydroisoquinoline;
4-(3,4-difluoro)phenyl-2-methyl-7-(3-pyridyl)-1,2,3,4-tetrahydroisoquinoline;
7-[(2-methoxy)-3-pyridyl]-2-methyl-4-phenyl-1,2,3,4-tetrahydroisoquinoline;
7-[(6-methoxy)-3-pyridyl]-2-methyl-4-phenyl-1,2,3,4-tetrahydroisoquinoline;
2-methyl-4-phenyl-7-(3,5-pyrimidyl)-1,2,3,4-tetrahydrolsoquinoline;
4-(3,4-difluoro)phenyl-2-methyl-7-(3,5-pyrimidyl)-1,2,3,4-tetrahydrolsoquinoline;
4-(4-methyl)phenyl-2-methyl-7-(3,5-pyrimidyl)-1,2,3,4-tetrahydroisoquinoline;
2-methyl-4-phenyl-7-(2,6-pyrimidyl)-1,2,3,4-tetrahydroisoquinoline;
7-(2,5-dimethyl-4-isoxazole)-4-(4-methoxy)phenyl-2-methyl-1,2,3,4-tetrahydroisoquinoline; and
4-(4-methoxy)phenyl-2-methyl-7-(2-pyridyl)-1,2,3,4-tetrahydroisoquinoline or an oxide thereof, a pharmaceutically acceptable salt thereof, a solvate thereof, or a prodrug thereof

24. A method according to claim 24, wherein the compound is the (+) stereoisomer.

25. A method according to claim 24, wherein the compound is the (−) stereoisomer.
